(19) United States
(12) Patent Application Publication
(10) Pub. No.: US 2002/0051779 A1
(43) Pub. Date: May 2, 2002

(54) Methods of thrombolytic organ treatment and repair

(75) Inventors: Frederick A. Gage, Baltimore, MD (US); Debra J. Battjes Siler, Johns Island, SC (US)

Correspondence Address: OLIFF & BERRIDGE, PLC, P.O. BOX 19928, ALEXANDRIA, VA, 22320, US

(73) Assignee: Organ Recovery Systems, Inc., Des Plaines, IL

(21) Appl. No.: 09/938,597

(22) Filed: Aug. 27, 2001

Related U.S. Application Data
(60) Provisional application No. 60/227,843, filed on Aug. 25, 2000.

(51) Int. Cl. A61K 38/48; C12N 5/08

(52) U.S. Cl. 424/94.64; 435/1.2

(57) ABSTRACT

The invention teaches methods and compositions for removing thrombi lodged in the microvasculature of an organ. To remove the thrombi, the organ may be perfused, flushed or washed with a suitable perfusion solution to which a sufficient amount of a thrombolytic agent, such as Streptokinase, has been added. The perfusing, flushing or washing process of the organ with the thrombolytic agent will promote thrombolysis on existing thrombi, prevent the formation of new thrombi in the organ, and/or open the vasculature of the organ thereby decreasing vascular resistance and increasing flow. The method of the invention may be practiced using an organ perfusion apparatus that would allow the viability of the organ to be sustained and/or restored upon perfusion with a thrombolytic agent.

This non-provisional application claims the benefit of U.S. Provisional Application No. 60/227,843 filed Aug. 25, 2000, the entire disclosure of which is hereby incorporated by reference.

### BACKGROUND OF THE INVENTION

1. Field of Invention

The invention relates to organ or perfusion. In particular, the invention relates to compositions and processes for organ perfusion with a thrombolytic agent, such as Streptokinase, to enhance the viability of the organ.

2. Description of Related Art

Ideally, organs would be procured in a manner that limits their warm ischemia time to essentially zero. Unfortunately, in reality, many organs are procured after extended periods of warm ischemia (i.e., 45 minutes or more). In addition to warm ischemic insult, microvascular alterations, including erythrocyte aggregation and thrombus formation, may occur, which also adversely impact the integrity of the organ.

Organs taken from non-heart-beating donors (NHBD) typically have been exposed to an extended period of warm ischemia, and the period of cardiac standstill has been quite significant. Thus, obstructions in the microvasculature from erythrocyte/leukocyte aggregates as well as microthrombi have formed. See Hansen et al., Transplant Proc. (1997) 29:3577; Kuroe et al., Eur. Surg. Res. (1991) 23:20. Experimental studies have implicated the formation of these obstructions results in the elevated vascular resistance of organs taken from NHBDs.

Under conditions of severe trauma, a heart-beating donor may begin to produce excess fibrinogen, which leads to a condition known as Disseminated Intravascular Coagulation (DIC). The excess fibrinogen in the blood is converted to insoluble fibrin gel that becomes lodged in the microvasculature of various organs. Although DIC can be diagnosed by evaluating the clotting factors along with the fluid input and output of the donor organ, it is not uncommon for the condition to be overlooked initially. Generally, DIC is not reported until the transplanting team or perfusionist examines the organs. At this stage, DIC is often diagnosed by the observation of petechia, which are minute red spots due to the rupture of capillaries, on the organ.

Once DIC has been diagnosed, the transplanting surgeon must make one of three choices regarding the disposition of the organ: a) transplant the organ, with the hope that the DIC is marginal, b) discard the organ, or c) perfuse the organ in an attempt to eliminate the DIC. In perfusing the organ, the occlusion of the blood vessels hampers the equilibration of the perfusate solution to the blood vessels of the organ. The current protocol for eliminating these blockages is to either increase the flow pressure, which could damage the organ, or include a vasodilator, such as Regitine (Phenatolamine), in an attempt to widen the vessels and flush the fibrin clots out. Unfortunately, high pressure perfusion (e.g., above about 60 mm Hg) can wash off the vascular endothelial lining of the organ and in general damages organ tissue, in particular at hypothermic temperatures where the organ does not have the neurological or endocrinal connections to protect itself by dilating its vasculature under high pressure.

In most cases, the fibrin clots are not successfully removed and the organ must be discarded. In cases where the perfused organ is transplanted, the effectiveness of the flush-out procedure is a major determinant for the later viability of the graft. See Yamauchi et al., Transplantation (May 2000) 69(9):1780-1784.

The perfusion of organs is generally performed using a perfusion machine and conducted at low temperature. Significant advances have been made in the design of organ perfusion apparatuses. See Daemen et al., Transplant International (1996); 9 Supplement 1:S76-80. The literature teaches that the low temperature machine perfusion of organs is preferred at low pressures with roller or diaphragm pumps delivering the perfusate at a controlled pressure. See Yland et al., Transplant International (1996); 9(6):535-540. Numerous control circuits and pumping configurations have been utilized to achieve this objective and to machine perfuse organs in general. See, for example, U.S. Pat. Nos. 5,338,662 and 5,494,822 to Sadri; U.S. Pat. No. 4,745,759 to Bauer et al.; U.S. Pat. Nos. 5,217,860 and 5,472,876 to Fahy et al.; U.S. Pat. No. 5,051,352 to Martindale et al.; U.S. Pat. No. 3,995,444 to Clark et al.; U.S. Pat. No. 4,629,686 to Gruenberg; U.S. Pat. Nos. 3,738,914 and 3,892,628 to Thorne et al.; U.S. Pat. Nos. 5,285,657 and 5,476,763 to Bacchi et al.; U.S. Pat. No. 5,157,930 to McGhee et al.; and U.S. Pat. No. 5,141,847 to Sugimachi et al.

Various perfusion solutions have also been developed in the art to address the need to restore or maintain an organ's physiological function after perfusion for an extended period of time at hypothermic temperatures. For example, U.S. Pat. No. 5,066,578 to Wikman-Coffelt discloses an organ preservation solution that contains large amounts of pyruvate. Wikman-Coffelt teaches that flooding of the organ with pyruvate bypasses glycolysis, the step in the cell energy cycle that utilizes adenosine triphosphate (ATP) to produce pyruvate, and pyruvate is then available to the mitochondria for oxidative phosphorylation producing ATP. Wikman-Coffelt teaches perfusing or washing an organ at a warm temperature with a first preservation solution containing pyruvate for removal of blood or other debris from the organ's vessels and to vasodilate, increase flow and load the cells with an energy supply in the form of a clean substrate, namely the pyruvate. The organ is then perfused with a second perfusion solution containing pyruvate and a small percentage of ethanol in order to stop the organ from working, vasodilate the blood vessels allowing for full vascular flow, continue to load the cells with pyruvate and preserve the energy state of the organ. Finally the organ is stored in a large volume of the first solution for 24 hours or longer at temperatures between 4° C. and 10° C.

Other solutions used for organ perfusion include: Collins solution, which consists predominantly of potassium phosphate, magnesium sulfate and glucose; a modified version of Collins solution called “EuroCollins,” in which the magnesium sulfate is omitted; University of Wisconsin solution (UW solution), in which much of the phosphate anion has been replaced with lactobionate, and in which glucose has been replaced with raffinose (which was found to provide better protection against adverse effects of cell swelling during hypothermic storage); and a modified version of UW solution called “Belzer Machine Perfusion Solution”. Other suitable solutions have been described, for example, in U.S. Pat. Nos. 5,643,712, 5,699,793, and 5,843,024 to Brasile and U.S. Pat. Nos. 5,599,659 and 5,702,881 to Brasile et al., as well as U.S. patent application Ser. No. 09/628,311 to Taylor, filed Jul. 28, 2000, each of which is incorporated herein by reference in its entirety. Each of these references describes separate resuscitation and preservation solutions for organs.

However, flooding an organ with these perfusates does not alleviate the problems caused by the formation of thrombi in the microvasculature of the organ. The blockages in the blood vessels formed by the thrombi would only impair the delivery of much needed oxygen as well as other nutrients in an organ stored at 20° C. or more. Further, assessment of the viability of an organ is necessary before the use of any type of solution can be determined to have been fruitful.

Thrombolytic drugs, such as Streptokinase; Urokinase; Alteplase, Tenecteplase (TNKase), or other recombinant tissue plasminogen activators (tPA); Anistreptase or other forms of anisoylated streptokinase; Reteplase, or other mutant tPAs, have been used in hospitals for rapid thrombolysis and to treat thrombotic disease. These proteins promote the degradation of thrombi by stimulating the conversion of endogenous plasminogen to plasmin, a proteolytic enzyme that hydrolyzes fibrin. However, the use of these agents has largely been limited to the treatment of acute thrombotic or enibolytic disease. Currently, thrombolytic agents are used for thrombolysis in the arteries of the heart, lungs or brain, in deep leg veins, or in indwelling intravenous catheters or artificial heart valves where thrombi may have formed. These agents are also used for the management of myocardial infarction in patients with established coronary arterial thrombosis and for the treatment of acute ischemic stroke . The most frequent adverse reaction associated with these agents is excessive bleeding.

More recently, Yamauchi et al., Transplantation (May 2000) 69(9): 1780-1784, have demonstrated in rats the benefits of pre-flushing a liver, taken from a non-heart-beating donor (NHBD), with Ringer's solution having up to 7,500 international units (I.U.) of Streptokinase, at 25° C. When the pre-flush was followed by perfusion with UW solution at 4° C., a marked improvement in graft perfusion was observed.

### SUMMARY OF THE INVENTION

Fibrin clots, excess fibrinogen and aggregated blood cells trapped in an organ's vasculature may prevent the organ from perfusing properly, or may cause the organ to function improperly, before and/or after transplantation. However, the problems caused by such substances may be prevented or alleviated by the present invention. Perfusion, diagnostic and transporter processes and apparatus of the invention provide ex vivo techniques that include perfusing, flushing or washing an organ with a perfusion solution containing suitable amounts of a thrombolytic agent to degrade thrombi that have formed, flush the degradation by-products out of the organ, prevent the formation of microthrombi in an organ, and to open the vasculature of the organ.

The present invention relates to compositions and methods for perfusing organs removed from a patient or donor and determined to have DIC, in order to remove fibrin clots lodged in the microvasculature of the organ. The present inventors have discovered that the addition of a thrombolytic agent such as Streptokinase to the organ perfusion solution, in suitable effective amounts, has been an effective therapeutic treatment for DIC during perfusion. This treatment may improve the viability of the perfused organ to a viability equivalent to non-DIC organs similarly perfused, but not requiring such a thrombolytic agent, thus enabling it to be transplanted.

The organ may be perfused, flushed or washed with a suitable perfusion solution to which a thrombolytic agent, such as Streptokinase, has been added. By perfusing, flushing or washing the organ with a perfusion solution containing a thrombolytic agent, thrombi that have formed can be degraded, flushed out of the organ, and/or the formation of new thrombi in the organ can be reduced or prevented. The method thus opens the vasculature of the organ permitting a more homogenous equilibration of the perfusion solution to the microvasculature of the organ. The resulting improvement in perfusion quality would improve the cold preservation of the organ, as well as the viability of the organ transplant. The method can also be used to minimize complications in organs removed from a patient that are later returned to the patient after the desired procedures have been performed.

The method can be practiced using any suitable perfusion, diagnostic, and/or transporter apparatus, such as those disclosed in U.S. patent application Ser. No. 09/645,525, filed Aug. 25, 2000, the entire disclosure of which is hereby incorporated by reference. These devices generally have the ability to detect the cell chemistry of an organ in order to adjust the perfusion parameters and control the cellular metabolism, for example to repair ischemic damage to the organ, to prevent reperfusion injury, to treat disease and/or treat damage to and/or enhance the properties of the organ. An advantage of such an apparatus is that it extends the time that an organ may be available for ex vivo treatment, e.g., for hours (e.g. 2-12 or more hours) or even days (e.g. 2-12 or more days) or weeks (e.g. 1-8 or more weeks).

The perfusion, diagnostic and/or transporter apparatus may be used to provide particular solutions or chemicals, such as thrombolytic agents, to an organ and may be used to perform particular treatments, including flushing or washing an organ with particular solutions or chemicals. Treatment with a thrombolytic agent and other ex vivo treatments may be performed on an organ to be transplanted or may be performed on an organ that has been removed from a patient and is to be returned to the patient after the desired procedure is performed.

Other ex vivo techniques and methods may be used individually and/or in conjunction with the methods and compositions of the invention, for example, to perform research on an organ. During the period in which the organ is preserved and/or maintained, various drug and other treatments for research and development may be performed on and/or with the organ.

### DETAILED DESCRIPTION OF THE INVENTION

The present invention provides a method of perfusing organs, removed from a patient or donor, to remove thrombi lodged in the microvasculature of the organ. The present invention also separately provides compositions, such as perfusion solutions, useful in such methods.

In embodiments of the present invention, a modified perfusion solution is provided. The modified perfusion solution includes a suitable conventional perfusion solution, with an effective amount of thrombolytic agent added thereto.

According to embodiments of the present invention, any suitable thrombolytic agent can be used. Suitable thrombolytic agents include, but are not limited to, Streptokinase; Urokinase; Alteplase, Tenecteplase (TNKase), or other recombinant tissue plasminogen activators (tPA); Anistreptase or other forms of anisoylated streptokinase; Reteplase, or other mutant tPAs, mixtures thereof, and the like. Any of the listed agents may be used, with no preference for any particular one. Selection may be made on the basis of availability, dosage desired, how supplied/packaged, ease of use, and conditions of use such as type of organ and desired perfusion temperature. These agents are primarily enzymes (proteins) and may be temperature-sensitive. Therefore, some agents may become less active at hypothermic temperatures. Product literature accompanying these agents should be consulted before use to verify the stability of the product following reconstitution, during storage, and at the desired perfusion temperature. Streptokinase is a convenient and widely available thrombolytic agent that retains its activity at low temperatures; however, any thrombolytic agent may be used.

The thrombolytic agent can be used alone or it can be added to any suitable solution or medium. Preferably, for purposes of dilution and ease of use, the thrombolytic agent is used in combination with, or as part of, a suitable solution. For example, where the thrombolytic agent is to be used for perfusing, flushing or washing an organ, the thrombolytic agent is reconstituted as directed and preferably mixed with or otherwise added to a suitable perfusion, flushing or washing solution. Any suitable perfusion solution can be used such as, but not limited to, ViaSpan™ (UW solution marketed by duPont), Belzer Machine Perfusion Solution (Belzer MPS available from Organ Recovery Systems), Custodial® (cardioplegia solution from Sangstat), EuroCollins, Lactated Ringers, Physiological Saline, or other crystalloid solutions containing oncotic agents such as dextran and HES (hydroxyethyl starch), solutions described in U.S. patent application Ser. No. 09/628,311, filed Jul. 28, 2000, the entire disclosure of which is hereby incorporated by reference, mixtures thereof, and the like. These solutions may also be used to wash or flush organs when perfusing an organ would not be practical.

When the thrombolytic agent is added to or otherwise mixed with a suitable solution, such as a perfusion solution, the thrombolytic agent can be incorporated in any suitable or effective amount. For example, in embodiments of the present invention, when used in the perfusion of an organ, the thrombolytic agent may be incorporated in an amount from about 5,000 or less to about 58,000,000 or more international units (I.U.), or from about 10 Units or less to about 30 Units or more, such as about 50, 100 or 200 Units. Reference Standards may be specific to the agent and may not be comparable with units used for other agents. However, the present invention is not limited to such amounts, and lesser or greater amounts can be used, as desired. As will be apparent, the dosage of thrombolytic agent used will vary according to the thrombolytic agent used, as well as other conditions such as, for example, the temperature and conditions of use and the volume of perfusate. Based on the disclosure of the present specification, one of ordinary skill in the art will be able to select appropriate amounts of specific thrombolytic agents for particular applications.

For example, in one embodiment of the present invention where Streptokinase is used, it may be used in any suitable amount of from about 5,000 or less to about 5,000,000 or more I.U., preferably from about 100,000 to about 400,000 I.U., and more preferably from about 25,000 or about 50,000 to about 450,000 or about 500,000 I.U. or from about 200,000 to about 300,000 I.U. Streptokinase is generally commercially available in bottles or vials of about 250,000, 750,000 or 1,500,000 I.U., and can be used as such or can be used or fractions or combinations of one or more such bottles or vials. In embodiments where Streptokinase is used in a flushing solution at temperatures of about 25° C., it is preferably used in amounts of 10,000 I.U. or more.

In other embodiments, such as where Urokinase is used, the amount can be preferably in a low range, such as 5,000 I.U. or less, or in a high range such as 250,000 I.U. or more. For example, Urokinase is generally commercially available in bottles or vials of about 5,000 I.U., such is generally used for catheter clearance, or in bottles or vials of 250,000 I.U., where recommended dosage is about 3 vials. Of course, the agent can be used as such or can be used or fractions or combinations of one or more such bottles or vials.

As other non-limiting examples, Reteplase is generally commercially packaged for administration of a 10 U dose. Anistreplase is generally commercially packaged in 30 Unit vials. Activase is generally commercially provided in either 50mg vials with 29 Million I.U., or 100 mg vials with 58 Million I.U.

Of course, varying amounts of any of the above-mentioned or other thrombolytic agents can be used according to the present invention. Thus, for example, under circumstances where perfusion of the organ is not possible, practical and/or desired, a greater amount of thrombolytic agent can be incorporated into the perfusion solution and the resultant solution can be used to wash or flush the organ.

The perfusion solutions according to the present invention can generally be used in any of the conventional or after developed perfusion, diagnostic, and/or transporter apparatus, such as those disclosed in U.S. patent application Ser. No. 09/645,525, filed Aug. 25, 2000, the entire disclosure of which is hereby incorporated by reference. According to processes of the present invention, the solution can be used in such perfusion apparatus for any suitable period of time. For example, the solution can be used in the apparatus for a period of time of from about 1 hour or less to about 20 hours or more, preferably from about 1 to about 20 hours, more preferably from about 3 to about 15 hours, and even more preferably from about 4 to about 12 hours, to provide the desired fibrinogen dissolution. The optimum time and temperature for perfusing an organ may be adjusted by routine experimentation in view of the present disclosure. However, in embodiments, the temperature may be between about 2° C. and about 10° C., preferably about 5° C. Because of the optimum temperature range of embodiments of the present invention, a perfusion solution optimized for hypothermic conditions (i.e., about 15° C. or lower) is preferred. However acceptable results may also be obtained according to the present invention by incorporating one or more thrombolytic agents into a solution that is optimized for different temperatures or conditions, such as for normothermic conditions.

According to the present invention, the perfusion method can be used to reduce or eliminate the number and/or size of thrombi in the organ being treated. Preferably, in the case where the organ has thrombi that are already formed, the perfusion is conducted for a sufficient time and under sufficient conditions to substantially eliminate the thrombi. Sufficient elimination of thrombi is indicated by the increase in flow rates and the decrease in vascular resistance, which would correlate with the degree of vascular clearance. Other observable indications of thrombolysis is the color of the effluent; the effluent will change to a bright red color as products of hemolyzed red blood cells are flushed out of the organ.

Furthermore, the perfusion process may be performed where the systolic pressure within the perfusion apparatus will not damage the vasculature of the organ. High-pressure perfusion (e.g., above about 60 mm Hg) can wash off the vascular endothelial lining of the organ and damage the organ tissue. This is a particular problem at hypothermic temperatures where the organ does not have the neurological or endocrinal connections to protect itself by dilating its vasculature in response to the high pressure.

The specific pressures, length of perfusion time and particular temperatures will vary depending on the particular organ or organs being perfused. For example, hearts and kidneys are preferably perfused at a pressure of approximately 10 to 100 mm Hg and a flow rate of approximately 3 to 5 ml/min. [ISN'T IT JUST ML/MIN?] for up to approximately 2 to 4 hours at normothermic temperatures. Perfusion within these parameters is designed to maintain and/or restore the viability of the organ by restoring and/or maintaining pre-ischemia energy levels of the organ. These organs are then preferably perfused at a pressure of approximately 10 to 30 mm Hg and a flow rate of approximately 1 to 2 ml/min. [ISN'T IT JUST ML/MIN?] for as long as approximately 72 hours to 7 days at hypothermic temperatures for storage and/or transport. However, these criteria will vary depending on the condition of the particular organ, the donor body and/or the donee body and/or on the size of the particular organ. One of ordinary skill in the art can select appropriate conditions without undue experimentation in view of the guidance set forth herein. Other organs that may be perfused according to the method of the invention may include, but are not limited to, the liver, pancreas, lungs and intestines.

In practicing the methods of the invention, the initial condition of the organ must be evaluated. The organ is checked, for example, for petechia, the number of vessels, the presence of any aortic plaque, or any other organ abnormalities. Once properly evaluated, the arteries in the organ are then cannulated with the proper sized cannula. The organ is then placed on the perfusion circuit where the circuit pressure is set to a suitable pressure, such as a systolic pressure of 45 mm Hg.

The organ may be perfused with a medical fluid, preferably synthetic, and may, for example, be a simple crystalloid solution, or may be augmented with an appropriate oxygen carrier. The oxygen carrier may, for example, be washed, stabilized red blood cells, cross-linked hemoglobin, pegolated hemoglobin or fluorocarbon based emulsions. The medical fluid may also contain antioxidants known to reduce peroxidation or free radical damage in the physiological environment and specific agents known to aid in tissue protection. An oxygenated (e.g., cross-linked hemoglobin-based bicarbonate) solution is preferred for normothermic perfusion while a non-oxygenated (e.g., simple crystalloid solution preferably augmented with antioxidants) solution is preferred for hypothermic perfusion.

In this initial perfusion of the organ, the perfusion solution used in either normothermic and hypothermic modes are designed to reduce or prevent the washing away of, or damage to, the vascular endothelial lining of the organ. For the hypothermic perfusion mode, as well as for flush and/or static storage, a preferred solution is the solution disclosed in U.S. patent application Ser. No. 09/628,311, filed Jul. 28, 2000, the entire disclosure of which is incorporated herein by reference. Examples of additives that may be used in perfusion solutions for the present invention are also disclosed in U.S. Pat. No. 6,046,046 to Hassanein, the entire disclosure of which is incorporated herein by reference. Of course, other suitable solutions and materials may be used, as is known in the art. The solutions can be modified to include one or more thrombolytic agents, as described above.

Preferably, to assist in determining the status and initial condition of a donor organ, the donor chart is reviewed for medically pertinent information in the donor's history. In addition, the hospital management of the donor and other pertinent donor information can preferably be reviewed. In particular, the donor chart is reviewed for the diagnosis of DIC or indications that DIC may be present. The key information used to diagnose DIC include an evaluation of the clotting factors (e.g., Prothrombin Time, or Plasma Thromboplastin Antecedent (coagulation factor XI or PTA)), which is often a component of standard liver enzyme tests, along with the fluid output and input of the organ.

The fluid input and output, as well as other fluid characteristics, such as organ resistance (pressure/flow), pH, PO₂, pCO₂, LDH, T/GST, T-protein, lactate, glucose, base excess and ionized calcium levels may be used to analyze and determine an organ's viability. The characteristics may be analyzed individually or multiple characteristics may be analyzed to determine the effect of various factors. The characteristics may be measured by capturing the venous outflow of the organ and comparing its chemistry to the perfusate inflow. The venous outflow may be captured directly and measured or the organ bath may be measured to provide a rough approximation of the fluid characteristics for comparisons over a period of time.

In an organ in which DIC has been diagnosed, the systolic pressure of the perfusion circuit can be increased, such as to 50 mm Hg, and a thrombolytic agent is added to the perfusion solution, as described above. For example, in embodiments of the present invention, 5,000 to 500,000 units, preferably 100,000 to 400,000 units, more preferably 200,000 to 300,000 units, and even more preferably about 250,000 units of a thrombolytic agent such as Streptokinase may be added to the perfusion solution or material. In such embodiments, the temperature for perfusing an organ may optimally be between about 2° C. and about 10° C., preferably about 5° C. However, different temperatures may be used, as will be apparent to one of ordinary skill in the art.

By perfusing, flushing or washing the organ with a thrombolytic agent, fibrin clots that have formed can be degraded and/or the formation of new clots in the organ can be prevented. This opens the vasculature of the organ and permits a more homogenous equilibration of the perfusion solution to the microvasculature of the organ. The resulting improvement in perfusion quality would improve the cold preservation of the organ, as well as the viability of the organ transplant. The method can also be used to minimize complications in organs removed from a patient that are later returned to the patient after the desired procedures have been performed.

Once the DIC has been reduced or preferably eliminated, the organ may be further processed for transplantation. The organ may be further processed for transplantation by one or more of hypothermic perfusion, normothermic perfusion, and/or static storage, in any necessary and/or desired order.

Alternatively, an organ treated according to the invention may undergo further ex vivo treatment by mechanical, physical, chemical or genetic manipulation and/or modification to treat disease and/or treat damage to and/or enhance the properties of the organ. An organ sample may be removed from a first body, modified, treated and/or analyzed outside the first body and either returned to the first body or transplanted to a second body. The advantage in treating the organ with a thrombolytic agent is that it can extend the time an organ may be available for ex vivo treatment, e.g., for hours (e.g. 2-12 or more hours) or even days (e.g. 2-12 or more days) or weeks (e.g. 1-8 or more weeks) without the adverse effects that blockages of the organ's microvasculature would cause.

Other ex vivo treatments may involve performing surgical techniques on an organ, such as cutting and suturing an organ, for example to remove necrotic tissue. Any surgical or other treatment technique that may be performed on an organ in vivo may also be performed on an organ ex vivo. The benefit of such ex vivo treatment may be seen, for example, in the application of radiation or chemotherapy to treat a tumor present in or on an organ. Ex vivo treatment prevents other portions of the patient from being subjected to extraneous radiation or chemotherapy during treatment. The methods and compositions of the present invention provide additional time for a physician to maintain the organ before, during and/or after performing a particular technique on the organ.

By way of Example only, and without being limited thereto, the method of the present invention is described as practiced on a human kidney. The kidney can be harvested from the donor under beating heart conditions. Following harvesting, the kidney can be flushed, such as with any suitable solution or material including, but not limited to ViaSpan™ (UW solution marketed by duPont), or other crystalloid solutions containing oncotic agents such as dextran, HES (hydroxyethyl starch), solutions described in U.S. patent application Ser. No. 09/628,311, filed Jul. 28, 2000, the entire disclosure of which is hereby incorporated by reference, or the like.

The method of the present invention is summarized below:

A. The kidney is evaluated, cannulated, and placed on a perfusion circuit.

1. The kidney is evaluated for the appearance of petechia, the number of vessels, the presence of aortic plaque, and any other vascular abnormalities,

2. The artery, or arteries, are cannulated with the proper sized cannula, and

3. The kidney is connected to perfusion circuit with a systolic pressure set to 45 mm Hg.

B. The donor chart is reviewed for:

1. Donor medical history,

2. Hospital management of donor, other donor information, and

3. Diagnosis of DIC.

C. If DIC is diagnosed, the systolic pressure in perfusion circuit is increased to 50 mm Hg, and 250,00 IU Streptokinase is added to the perfusion solution.

D. The perfusate is recirculated through the kidney at 5° C.

E. The kidney is perfused for 4-12 hours to degrade the fibrin clots.

F. The kidney may be further processed for transplant after the DIC has been eliminated.

The above described method may be used for child or small organs as well as for large or adult organs with modification as needed of the pressures and flow rates accordingly. Once the clots and the degradation by-products have been flushed from the organ, the viability of the organ can be monitored, and the disposition of the organ can be determined.

### EXAMPLE

A kidney is treated with 250,000 units of Streptokinase when one or more of the following donor evaluation markers is present: written documentation of DIC or other coagulation problems in the donor's chart, large differences in the fluid balance of the donor (input versus output), the use of Pitressin, or the appearance of petechia on the kidney.

The kidney is biopsied and cannulated following standard protocols. The kidney is placed into the organ preservation circuit and a perfusion technician monitors the pressure, output flow, calculated vascular resistance, osmolarity, pH, pCO₂, PO₂, K⁺, and base excess of the organ for a minimum of 30 minutes to get baseline data. A bolus of 250,000 units of Streptokinase (reconstituted lyophilized powder) as a thrombolytic agent is injected into the perfusion circuit and monitoring of the above variables continues.

The initial flow and vascular resistance are measured prior to adding Streptokinase to the perfusate solution. The perfusate's normal color and opacity is clear with a yellow tint. However, after the kidney receives the Streptokinase bolus, the perfusate changes to a bright red color, similar to the appearance of arterial blood. Over a period of time, there appears to be red cell sediment on the floor of the arterial reservoir. The vascular resistance of the kidney decreases and output flow increases over time as the Streptokinase promotes thrombolysis. The flow and vascular resistance are then measured at 1 hour and 4 hours after the addition of Streptokinase to the perfusate solution. The final measures of flow and vascular resistance are taken immediately prior to the removal of the kidney from the perfusion circuit. Table 1 summarizes the effects on flow rates and vascular resistance after treatment according to the above protocol.

TABLE 1
Flow Rates and Vascular Resistance in Six Groups of Kidneys
(Values presented are the Mean + SEM)

| | FLOW (ml/min) | | RESISTANCE (R Units) | |
|---|---|---|---|---|
| | Start Point | End Point | Start Point | End Point |
| TRANSPLANTED KIDNEYS | | | | |
| DIC-Treated; n = 14 | 98.6 ± 3.7 | 145.0 ± 7.6 | 0.345 ± 0.015 | 0.217 ± 0.017 |
| DIC-Untreated; n = 0 | — | — | — | — |
| Non-DIC; n = 14 | 86.9 ± 9.04 | 142.4 ± 7.6 | 0.379 ± 0.030 | 0.187 ± 0.015 |
| MEDICALLY | | | | |
| DISPOSED KIDNEYS | | | | |
| DIC-Treated; n = 10 | 56.0 ± 6.1 | 86.1 ± 6.3 | 0.810 ± 0.125 | 0.465 ± 0.059 |
| DIC-Untreated; n = 9 | 58.6 ± 6.0 | 81.0 ± 12.3 | 0.658 ± 0.062 | 0.469 ± 0.066 |
| Non-DIC; n = 10 | 59.3 ± 9.6 | 96.1 ± 7.9 | 0.796 ± 0.143 | 0.361 ± 0.033 |

Definition of Groups:

DIC-Treated: Kidneys diagnosed with DIC and perfused with Streptokinase added to the preservation solution

DIC-Untreated: Kidneys diagnosed with DIC and perfused without Streptokinase added to the preservation solution

Non-DIC: Kidneys that did not have DIC and were perfused without Streptokinase added to the preservation solution (Controls)

Generally, the unsuitability of a kidney for transplant is based on either abnormal biopsy results (as previously discussed) and/or perfusion parameters remaining outside of acceptable limits. Although the limits are not absolute, kidneys that are acceptable for transplant are expected to exhibit flow rates greater than 100 ml/min and have a vascular resistance less than 0.400 R Units. Kidneys that do not meet these criteria may be “Medically Disposed” or discarded because they are deemed unsuitable for transplant.

Comparisons of the start points and end points between the three groups of medically disposed kidneys shows that the flow rates and resistance values do not differ significantly between the organs of the three groups. A comparison of the start points of the two groups of kidneys that are subsequently transplanted shows that these two groups are also not significantly different for either flow rate or vascular resistance.

However, of note is the comparison of the end points between the transplanted DIC-Treated kidneys and the transplanted Non-DIC kidneys, which also shows little significant difference in measures of flow rate and resistance. This result demonstrates that Streptokinase treatment of the DIC kidneys enables them to respond to the perfusion process in a manner similar to normal, non-DIC kidneys, and brings flow and resistance to an acceptable endpoint. Therefore, in this Example, the treatment enabled the transplant of 14 kidneys that would otherwise have been discarded.

A similar comparison of the end points between the transplanted DIC-Treated kidneys and the DIC-Untreated kidneys (all of which had to be discarded), however, shows statistically significant differences in flow rate and resistance measures (p<0.0001 for flow and p=0.0002 for vascular resistance). Also significant is the difference between the end points of the DIC-Untreated and the Non-DIC kidneys (p=0.0002 for flow and p<0.0001 for resistance). These results clearly demonstrate that thrombolytic treatment of DIC kidneys is effective and necessary if these normally discarded kidneys are to be considered potentially transplantable.

The various characteristics of the kidneys used in this example and the ultimate disposition of the kidneys are summarized in Table 2.

TABLE 2

| | DIC-TREATED | DIC-UNTREATED | CONTROL |
|---|---|---|---|
| | (n = 13 donors, | (n = 5 donors, | (n = 14 donors, |
| CHARACTERISTIC | 24 kidneys) | 9 kidneys) | 24 kidneys) |
| Age Range | 15-73 | 57-78 | 16-76 |
| Average Age (yrs) | 36.7 | 67.4 | 35.6 |
| Gender-Men | 6 | 3 | 6 |
| Gender-Women | 7 | 2 | 7 |
| Race-Caucasian | 11 | 4 | 11 |
| Race-Hispanic | 1 | 0 | 1 |
| Race-African American | 1 | 1 | 1 |
| Cause of Death-MVA | 8 | 0 | 7 |
| Cause of Death-ICB | 3 | 5 | 3 |
| Cause of Death-GSW | 1 | 0 | 1 |
| Cause of Death-Other | 1 | 0 | 3 |
| Total Preservation Time (hrs) | 11.80-43.26 | 11.88-26.67 | 17.20-47.17 |
| Average (hrs) | 26.43 | 19.25 | 31.93 |
| Number Kidneys in Group | 24 | 9 | 24 |
| Number Transplanted | 14 (58%) | 0 | 14 (58%) |
| Number Medically Disposed | 10 (42%) | 9 (100%) | 10 (42%) |

MVA = Motor Vehicle Accident
ICB = Intracranial Bleed
GSW = Gun Shot Wound
Total Preservation Time = Kidney Cross-Clamped in Donor to Re-establish flows in Recipient

Although treatment of donor organs with thrombolytic agents may be successful in eliminating DIC, the organ may still have to be discarded following microscopic examination of biopsies taken prior to perfusion. Changes in the vasculature caused by hypertension, evidence of early onset of diabetes, or, in the particular case of kidneys, sclerosis of the renal glomeruli are examples of conditions that may render an organ unsuitable for transplant.

While the invention has been described in conjunction with specific embodiments thereof, it is evident that many alternatives, modifications and variations may be apparent to those skilled in the art. Accordingly, the preferred embodiments of the invention as set forth herein are intended to be illustrative, not limiting. Various changes may be made without departing from the spirit and scope of the invention.

What is claimed is:

1. A method of treating an organ with a thrombolytic agent to promote thrombolysis or prevent the formation of new thrombi, comprising perfusing said organ with a perfusion solution comprising a thrombolytic agent.

2. The method according to claim 1, wherein the organ is an organ removed from a human.

3. The method according to claim 1, wherein said perfusion comprises:
a) connecting said organ to a perfusion circuit, and
b) recirculating the perfusion solution through the organ.

4. The method according to claim 1, wherein the perfusion is conducted until it appears that thrombolysis is substantially complete and measured parameters are within acceptable limits.

5. The method according to claim 1, wherein the thrombolytic agent is selected from the group consisting of Streptokinase; Urokinase; Alteplase, Tenecteplase, other recombinant tissue plasminogen activators, Anistreptase, anisoylated streptokinase, Reteplase, and other mutant tPAs.

6. The method according to claim 1, wherein the thrombolytic agent is Streptokinase.

7. The method according to claim 6, wherein an amount of the thrombolytic agent used is between 10,000 to 1,500,000 IU.

8. The method according to claim 6, wherein an amount of the thrombolytic agent used is between 100,000 to 300,000 IU.

9. The method according to claim 1, wherein an amount of the thrombolytic agent used is from about 5,000 to about 58,000,000 IU, or from about 10 to about 30 or more Units.

10. The method according to claim 1, wherein an amount of the thrombolytic agent used is about 250,000 IU.

11. The method according to claim 1, wherein the perfusion solution further contains a vasodilator.

12. The method according to claim 3, wherein the perfusion circuit has a systolic pressure of less than 60 mm Hg.

13. The method according to claim 3, wherein the perfusion circuit has a systolic pressure between 45 mm Hg and 60 mm Hg.

14. The method according to claim 3, wherein the perfusion circuit has a systolic pressure of about 50 mm Hg.

15. The method according to claim 3, wherein the perfusion solution is recirculated at a temperature between 2° C. and 10° C.

16. The method according to claim 3, wherein the perfusion solution is recirculated at a temperature of about 5° C.

17. The method according to claim 1, wherein the organ is perfused for 1 to 20 hours.

18. The method according to claim 1, wherein the organ is perfused for at least 4 hours.

19. The method according to claim 1, wherein the organ is perfused for 4 to 12 hours.

20. The method according to claim 1, wherein the organ is selected from the group consisting of heart, liver, kidney, lung, pancreas and intestine.

21. A method of treating a kidney with a thrombolytic agent to promote thrombolysis or prevent formation of new thrombi, comprising perfusing said kidney with a perfusion solution comprising a thrombolytic agent.

22. The method according to claim 21, wherein the kidney is a kidney removed from a human.

23. The method according to claim 21, wherein said perfusion comprises:
a) connecting said kidney to a perfusion circuit, and
b) recirculating the perfusion solution through the kidney.

24. The method according to claim 21, wherein the perfusion is conducted until it appears that thrombolysis is substantially complete and measured parameters are within acceptable limits.

25. The method according to claim 21, wherein the thrombolytic agent is selected from the group consisting of Streptokinase; Urokinase; Alteplase, Tenecteplase, other recombinant tissue plasminogen activators, Anistreptase, anisoylated streptokinase, Reteplase, and other mutant tPAs.

26. The method according to claim 21, wherein the thrombolytic agent is Streptokinase.

27. The method according to claim 26, wherein an amount of the thrombolytic agent used is between 10,000 to 1,500,000 IU.

28. The method according to claim 26, wherein an amount of the thrombolytic agent used is between 100,000 to 300,000 IU.

29. The method according to claim 21, wherein an amount of the thrombolytic agent used is from about 5,000 to about 58,000,000 IU, or from about 10 to about 30 or more Units.

30. The method according to claim 21, wherein the kidney is perfused for 1 to 20 hours.

31. The method according to claim 21, wherein the kidney is perfused for 4 to 12 hours.

32. The method according to claim 22, wherein the perfusion solution is recirculated at a temperature of about 5° C.

33. The method according to claim 22, wherein the perfusion circuit has a systolic pressure greater than 45 mm Hg and less than 60 mm Hg.

34. The method according to claim 22, wherein the perfusion circuit has a systolic pressure of about 50 mm Hg.

35. A solution for perfusing, washing, or flushing an organ, comprising:
a) a perfusion solution; and
b) a thrombolytic agent,
provided that when said thrombolytic agent is Streptokinase, said Streptokinase is used in an amount of at least about 10,000 IU.

36. The solution according to claim 35, wherein the perfusion solution is optimized for a hypothermic mode of an organ perfusion apparatus.

37. The solution according to claim 35, wherein the thrombolytic agent is selected from the group consisting of Streptokinase; Urokinase; Alteplase, Tenecteplase, other recombinant tissue plasminogen activators, Anistreptase, anisoylated streptokinase, Reteplase, and other mutant tPAs.

38. The solution according to claim 35, wherein the thrombolytic agent is Streptokinase.

39. The method according to claim 38, wherein an amount of the thrombolytic agent used is between 10,000 to 1,500,000 IU.

40. The method according to claim 38, wherein an amount of the thrombolytic agent used is between 100,000 to 300,000 IU.

41. The method according to claim 35, wherein an amount of the thrombolytic agent used is from about 5,000 to about 58,000,000 IU, or from about 10 to about 30 or more Units.

42. The solution according to claim 35, wherein the perfusion solution comprises any suitable organ preservation medium that provides ionic and oncotic support during perfusion.

43. The solution according to claim 35, wherein the perfusion solution further comprises a vasodilator.
